(19) United States
(12) Patent Application Publication
(10) Pub. No.: US 2012/0294836 A1
(43) Pub. Date: Nov. 22, 2012

(54) HIGH YIELD METHOD AND APPARATUS FOR VOLUME REDUCTION AND WASHING OF THERAPEUTIC CELLS USING TANGENTIAL FLOW FILTRATION

(75) Inventors: Jonathan ROWLEY, Walkersville, MD (US); Jacob PATTASSERIL, Mt. Airy, MD (US); Ali MOHAMED, Boyds, MD (US)

(73) Assignee: LONZA WALKERSVILLE, INC., Walkersville, MD (US)

(21) Appl. No.: 13/552,834

(22) Filed: Jul. 19, 2012

Related U.S. Application Data
(63) Continuation-in-part of application No. PCT/US2011/022054, filed on Jan. 21, 2011.
(60) Provisional application No. 61/297,368, filed on Jan. 22, 2010.

(51) Int. Cl. A61K 35/12; C12Q 1/02; C12N 5/071

(52) U.S. Cl. 424/93.7; 435/325; 435/29; 435/374

(57) ABSTRACT

The present invention provides processes for aseptically processing live mammalian cells in an aqueous medium to produce a cell suspension having a cell density of at least about 10 million cells/mL and cell viability of at least about 90%. These methods comprise a step of reducing the volume of the medium using a tangential flow filter (TFF) having a pore size of greater than 0.1 micron, during which step the trans-membrane pressure (TMP) is maintained at less than about 3 psi and the shear rate is maintained at less than about 4000 sec⁻¹. The invention also provides a complete process for large scale manufacturing mammalian cells for use in a therapeutic composition, and scalable, fully disposable systems for carrying out the process, using readily available disposables and pumps.

### CROSS-REFERENCE TO RELATED APPLICATIONS

This application is a continuation-in-part of PCT/US2011/022054, filed Jan. 21, 2011, which claims the benefit of U.S. Provisional Patent Application No. 61/297,368, filed Jan. 22, 2010, the contents of each of which are incorporated herein by reference.

### FIELD OF THE INVENTION

The present invention relates to methods and apparatus for manufacturing somatic cell therapy products that comply with regulatory agency requirements, such as current good manufacturing practice (cGMP) regulations for devices, biologics and drugs. More in particular, the present invention relates to processes and apparati for aseptically concentrating and washing live mammalian cells using Tangential Flow Filtration (“TFF”), particularly live mammalian cells that are used in a therapeutic product.

### BACKGROUND OF THE INVENTION

The FDA defines cell therapy as the prevention, treatment, cure or mitigation of disease or injuries in humans by the administration of autologous, allogeneic or xenogeneic cells that have been manipulated or altered ex vivo. The goal of cell therapy, overlapping that of regenerative medicine, is to repair, replace or restore damaged tissues or organs.

Ex vivo expansion of cells obtained from human donors is being used, for example, to increase the numbers of stem and progenitor cells available for autologous and allogeneic cell therapy. For instance, multipotent mesenchymal stromal cells (MSCs) are currently exploited in numerous clinical trials to investigate their potential in immune regulation, hematopoiesis, and tissue regeneration. The low frequency of MSCs necessitates cell expansion to achieve transplantable numbers.

The challenge for any cell therapy is to assure safe and high-quality cell production. In particular, cell processing under current Good Manufacturing Practice (cGMP)-graded conditions is mandatory for the progress of such advanced cell therapies. For allogeneic therapies, the economics of testing and certification of processes and products for GMP compliance are a significant cost factor in cell manufacturing, strongly encouraging production of maximum batch size and minimum batch run.

Optimally, therefore, therapeutic cell manufacturing for clinical-scale expansion would be conducted in a completely automated closed process from cell collection through post-culture processing. Such a closed process would facilitate cGMP-compliant manufacturing of cell therapy products in a form suitable for storage and ready for use in a clinical setting, with minimal risk of microbial contamination and viability losses due to mechanical or physiological stress. Some systems for such closed processes have been developed for relatively small-scale production of autologous cell therapy products (see, e.g., U.S. Pat. App. Pub. No. 2008/0175825 by Hampson et al.), but for various reasons such systems are not readily scaled for larger preparations, for instance, as anticipate for allogeneic products.

Large-scale automated, closed processes for use of mammalian cells to manufacture proteins, such as biotherapeutics, are well established. However, most such processes are designed to recover a protein product and discard the cells under conditions leading to cell death, either intentionally, as when cells are disrupted to release of intracellular products, or incidentally, when cells are separated from secreted products by harsh methods such as high speed centrifugation. In contrast, processing of therapeutic cells after expansion typically requires cell harvesting, volume reduction, washing, formulation, filling of storage containers and, often, cryopreservation of the product cells, all under conditions maintaining cell viability, biological functionalities and, ultimately, clinical efficacy.

In addition, therapeutic cells may not survive known processes for handling cells used for protein production because the latter typically represent highly-manipulated cell lines which, during extensive replication in culture, may have undergone selection for less sensitivity to mechanical shear forces and physiological stresses than exhibited, for instance, by progenitor or stem cells used in cell therapies. Thus, to retain efficacy, therapeutic cells typically are minimally cultured so as to maintain the original parental phenotype displayed upon isolation from human tissue; and hence, therapeutic cells generally are not selected or genetically engineered to facilitate downstream processing.

As technologies are developed to scale the cell culture processes, the technology required for downstream processing has quickly been overwhelmed. Specifically, volume reduction and washing of large amounts (e.g., 10-100 liters) of therapeutic cell suspensions with current technologies are time consuming and not scalable. Current technology, such as open centrifugation, may require 4-8 hours by 5-20 highly trained technicians using tens to hundreds of individual processing vessels, thus increasing manipulations and risk of contamination. Much of the field of cell therapy utilizes small scale blood processing equipment, which cannot be scaled to more than about ten liters per process. Thus, processing time and labor, and production costs are major constraints to be resolved in therapeutic cell volume reduction and washing, and there are further benefits to process equipment that can scale from the 5-10 liter range to several hundred liters, while at the same time maintaining the critical quality parameters of the process and resulting cell product. Such critical quality parameters include: cell suspension densities sufficient for therapeutic formulations (e.g., greater than 10 million cells/mL in most cases, and at least 30-70 million cells in some cases): high viability of the final cell product (e.g., greater than 90%) to maintain functionality and safety: high yield of cells (e.g., greater than 90% of the starting cells) to minimize loss of the high value cells; and reduction of residual levels of harvest reagents (e.g., trypsin or other enzyme) and media components (e.g., serum components, active growth factors, and the like) to acceptable levels for regulatory purposes.

Accordingly, there is a need for improved processes for manufacturing therapeutic cells, from cell collection through post-culture processing, including processes for efficient volume reduction and washing of cell suspensions with high yields of viable cells and low residual levels of culture or processing components that are detrimental to therapeutic use of the cells, particularly such processes that facilitate manufacturing in automated, closed systems.

U.S. Pat. No. 5,053,334 (“the '334 patent”) on a “Process for Producing Biologically Active Plasminogen Activator in Recombinant CHO Cells Using Suspension Culture and Removing Detrimental Components from Medium,” issued to Arathoon et al. on Oct. 1, 1991, discloses a method of producing biologically active human tissue plasminogen activator in suspension culture is provided wherein recombinant Chinese hamster ovary (CHO) cells are cultured and certain components detrimental to recovery and biological activity are removed as a cell-free filtrate by cross-flow filtration. The '334 patent exemplifies the disclosed process with a suspension of CHO cells in medium comprising fetal bovine serum (2% v/v) at a population density of about one million cells/mL. See “Examples,” The cells were subjected to medium exchange before being resuspended in serum-free production medium for about 90 hours. The medium exchange was effected using a hollow fiber tangential flow filter with 0.1 micron pores and a filtration area of 4.15 ft² with removal of filtrate at a rate of about 211 mL per minute, thereby reducing the culture volume to approximately 5.2 liters. At this time fresh sterile serum-free medium was pumped into the culture vessel at a rate of about 211 mLs per minute thus maintaining the retentate volume while constantly diluting out the old medium. Fifty-five liters of fresh serum free medium were pumped through the system to give a calculated reduction in serum concentration of about 190,000 fold (or less than 0.0001% by volume) when an aliquot of the cell suspension was added to fresh serum free medium in a separate 10 L stainless steel fermenter. The size of the aliquot added to the 10 L of fresh medium (and hence the associated dilution factor) was not disclosed; and quantitative viability of the cells was not reported.

U.S. Pat. No. 5,256,294 (“the '294 patent”) on a “Tangential Flow Filtration Process and Apparatus,” issued to van Reis on Oct. 26, 1993, discloses processes and apparati for separating species of interest from a mixture containing them which comprises subjecting the mixture to tangential-flow filtration, wherein the filtration membrane preferably has a pore size that retains species with a size up to about 10 microns, and the flux is maintained at a level ranging from about 5% up to 100% of transition point flux.

Trinh, L. and Shiloach, J., “Recovery of insect cells using hollow fiber microfiltration,” Biotechnol Bioeng. 48(4):401-405 (1995), discloses a method for media separation and cell collection for eukaryotic cells growing in suspension. The method is based on tangential flow microfiltration using an open channel arrangement in a hollow fiber configuration. See Abstract. Best results (highest processing flux rate) for polysulfone hollow fibers were reported using fibers with internal diameter of 0.75 mm, 0.45 micron pore size, and a cell suspension flow at a shear rate of 14000ₛ⁻¹ (0.032 L/min per fiber). The authors reported that a flux rate of 500 L/m² h can be obtained by maintaining the surface area/cell ratio at 0.05 m²/10 L of cells at a concentration of 2.5×10⁶ cells/mL. Forty liters of infected insect cells were the to be concentrated 10 fold in 20 min without affecting cell viability.

U.S. Pat. No. 6,068,775 (“the '775 patent”) on “Removal of Agent From Cell Suspension,” issued to Custer et al. on May 20, 2000, discloses a method of removing an agent (e.g., DMSO) from a suspension of cells using a semi-permeable membrane. In one aspect of the invention, the cells are used to bioprocess a biological fluid after removal of the agent. Volume reduction of a cell suspension is not disclosed.

U.S. Pat. No. 6,607,669 (“the '669 patent”) on “Method and Apparatus for Enhancing Filtration Yields in Tangential Flow Filtration,” issued on Aug. 19, 2003 to Schick, discloses a system for proceeding with filtration of liquids in a manner having enhanced control characteristics in which yields are said to be enhanced. See Abstract. The system and method can be used to maintain a substantially constant trans-membrane pressure. When desired, that constant trans-membrane pressure is especially well-suited to yield enhancement for the particular liquid being filtered, concentrated or collected, while minimizing a risk of damage to or loss of valuable components. Additionally, a constant feed rate or pump output can be maintained. The '669 patent further discloses that one object of the invention is to provide an improved apparatus and method for exacting filtration of liquids through a constant pressure mode which enhances yield of collected components.

Use of the system of the '669 patent is exemplified by a TFF process for separating extracellular protein (IgG) from a suspension of cells. See Example 1. More in particular, a 500 liter suspension of Chinese Hamster Ovary (CHO) cells at 3 million cells/mL was concentrated with a filtration unit have a membrane having 0.45 micron pores and a surface area of 0.6 m², with a constant trans-membrane pressure (TMP) of 5.0 psi. Cell-free media was collected as permeate, while the retentate contained the cell suspension, which was said to become increasingly concentrated as the filtration progressed. The volume of the cell suspension was reduced 250 times, down to 2.0 liters over a 3.5 hour processing time period, with an average system flux (permeate volume flow) over the 3.5 hours of 237 liters/hr-m² at the TMP of psi. The '669 patent reports that “[t]he concentration of the cell suspension was accomplished without affecting the viability of the cells, which was confirmed by the successful utilization of the cells in a subsequent procedure.” However, no data on yield of cells or quantitation of viability are reported.

TFF also has been employed for size-based separation of monocytes (see e.g., U.S. Pat. App. Pub. No. 2005/0173315) or CD34+ stem cells (U.S. Pat. App. Pub. No. 2005/0189297) from blood or bone marrow. These processes use TFF membranes of large pore sizes (e.g, 1-10 microns are claimed) where the raw materials (blood or bone marrow) are run through the filter and the cells of interest are increased in percentage in the retentate (4.2% CD34+ cells increased to 18% from bone marrow, 32% monocytes, to 71% in blood). These cell-based processes separate cells of various sizes using TFF, but do not disclose critical quality parameters of cell-based products such as percentage viability of separated cells, total viability of the final cell suspension, or biological functionality of these separated cells. Furthermore, they do not discuss the yields from these processes in terms of percentage of initial cells, and the disclosed processes are applicable to small scale processing only, whereas processing of tens to hundreds of liters of cell suspensions is not taught.

### SUMMARY OF THE INVENTION

The present invention provides processes and apparati for aseptically concentrating and washing live mammalian cells using Tangential Flow Filtration (“TFF,” also known as Cross-Flow Filtration (“CFF”)). The invention is particularly useful for live mammalian cells that are used in a therapeutic product, such as for volume reduction and washing of suspensions of such cells for formulation for cryopreservation or for administration to a subject.

In particular, the invention provides a high yield process for TFF that allows for volume reduction and washing of tens or hundreds of liters of harvested mammalian cells to create cell compositions suitable for pharmaceutical formulation at least about 5 million cells/mL with at least 90% yield of starting cells and at least 90% viability of the final cell product. The pharmaceutical compositions from this process have residual levels of components detrimental to therapeutic use (e.g., serum components, harvesting reagents such as trypsin or other enzymes) decreased by at least about one thousand to ten thousand fold compared to starting levels, allowing reduction to final levels below one part per million as required for serum, for instance, in certain biological preparations for therapeutic use (21 CFR §610.15(b)). This process has been reduced to practice, for instance, in a single use apparatus using steps to minimize shear rates, maximize flux (volume/surface area×time, e.g., L/m² h or “LMH” herein), and to minimize processing time—which all contribute to high quality cell suspensions suitable for human administration. The process is dependent on filter pore size, and is relatively independent of filter type (hollow fiber versus flat sheet), filter material, and buffer formulation. Other process variations have been employed and optimized to obtain cell suspensions ranging from 5-60 million cells/mL, within processing times of 1-3 hours. Operative variations are described which can be utilized to obtain very fast processing times (maximized permeate flux) and very high cell concentrations (e.g., greater than 30 million cells/mL).

Thus, one aspect of the present invention provides a method for aseptically processing live mammalian cells in an aqueous medium to produce a cell suspension having a cell density of at least about 5 million cells/mL and cell viability of at least about 80%. This method comprises a step of reducing the volume of the medium using a tangential flow filter (TFF) having a pore size of greater than 0.1 micron. During this step, to maintain desirable trans-membrane flux rates, the trans-membrane pressure (TMP) is maintained relatively low compared to prior art conditions for TFF processing of protein solutions. For instance, the TMP is maintained at less than about 5 psi, preferably at less than about 3 psi, and more preferably at less than 1 psi. During this processing step of the invention, to maintain cell viability, the shear rate is also maintained relatively low compared to prior art conditions for protein solutions, for instance, at less than about 4000 sec⁻¹, preferably at less than about 3000 sec⁻¹, and more preferably at less than about 2000 sec⁻¹.

In this method of the invention, to obtain desirable transmembrane flux rates, it is generally advantageous for the pore size of the TFF to be at least about 0.1 micron, preferably greater than 0.1 micron and more preferably about 0.65 micron. The TFF in this method may be of any configuration suitable for the desired processing volume, such as hollow fiber or sheet configurations, which are generally known in the art. Preferably, for processing about 10 L to about 100 L of cells according to the invention, the TFF is a hollow fiber filter with a surface area of about 0.5 ft². The number of cells processed per square foot of TFF area according to the present invention may be at least as low as 0.75 billion cells and at least as high as about 18 billion cells, while still maintaining the desired operational parameters such as cell quality and transmembrane flux rates.

Surprisingly, the relatively low sheer and TMP parameters of the present invention method do not result in “clogging” of the filter and, instead, produce highly desirable transmembrane flux rates, such as at least about 50 L/m² h, preferably at least about 100 L/m² h, more preferably at least about 200 L/m² h, and more preferably at least about 300 to about 600 L/m² h. Such flux rates greatly reduce the processing time for cell batch volumes of about 10 L to about 100 L, or even larger batches up to at least about 1000 L, compared to conventional centrifugation methods, thereby maintaining high cell quality including viability.

Further, the above TFF process parameters of the invention surprisingly provide excellent recovery of cells in the resulting cell suspension, for instance, at least about 80% to about 85%, preferably at least about 90% to about 95%, and more preferably at least about 97% to 100%, of the starting cells in the aqueous medium. Depending on the type of cell and the intended use, the resulting cell suspension may contain at least about 5 million, 10 million, 25 million, 50 million or at least about 75 million viable cells/mL, and in some cases densities of over about 100 million cells/mL are possible.

In some embodiments, the invention method further comprises a diafiltration step in which the TFF is used to wash the cells in the resulting suspension with a volume of an aqueous wash medium equal to at least about 2-4, preferably at least about 4-6 and more preferably at least about 8-10 times the volume of the cell suspension. The diafiltration step of the invention may reduce the residual level of an undesirable soluble component in the cell suspension by at least about 300 fold, preferably by about 1000 fold, and more preferably by about 3000 fold, compared to the level in original aqueous medium containing the cells. In practice, for instance, this step can reduce the residual level of culture medium components such as serum protein (e.g., BSA or HSA), or of harvesting reagents, such as trypsin, to less than about one part per million of final cell suspension, as required, for instance, in certain biological preparations for therapeutic use (21 CFR §610.15(b)).

In another aspect, the invention provides a complete process for manufacturing mammalian cells for use in a therapeutic composition. This method comprises the following steps: expanding the cells using any known large scale cell culture methodology, for instance, 10 layer or 40 layer vessels (“Cell Factories”), or a “wave” agitated bag or stirred tank bioreactor; harvesting the cells in a aqueous medium; and reducing the volume of the cells in the aqueous medium and washing the cells using the TFF method of the invention with a flat sheet or hollow fiber TFF configuration. This invention process further comprises formulating the resulting cell suspension in a cryoprotective medium and freezing and storing the formulated cells under conditions suitable for long-term maintenance of cell viability, using cryopreservation technology known in the art (e.g., involving dimethylsulfoxide (DMSO) as a cryoprotectant). The cells in cryprotective formulation may be frozen and stored in any conventional container known for such purpose, such as a plastic bag or glass vial, and stored for short periods at a temperature of at least about −80° C., or for long term storage, in the vapor or liquid phase of liquid nitrogen.

This manufacturing process of the invention produces frozen formulated cells which exhibit the following parameters: cell viability on thawing of at least about 80%; 90% or 95%; viable cell concentration greater than about 1 million, 3 million or 6 million cells/mL; and residual levels of an undesirable protein component reduced by at least about 1000 fold compared to the level in the initial aqueous medium, to a level of less than about 1 ppm.

An additional aspect of the present invention relates a completely closed, fully disposable and scalable Tangential Flow Filtration (TFF) system in the cell processing method of the invention, using, for instance, a Hollow-Fiber Filter (HFF). This TFF system can process (separate, clarify, recover and collect cells from the fluid media) large volume batches in less than 3 hrs while maintaining high cell viability and functionality. In this system the HFF has aseptic quick connectors attached such that system assembly simply requires three quick connections and sterile welds of plastic tubing (depending on batch size). FIG. 1 below shows an exemplary TFF system of the present invention, completely closed and fully disposable, comprising tubing, disposable sensors (P1, P2, and P3 in FIG. 1) and processing reservoir (FIG. 1) as well as a disposable filter, all commercially available. The TFF system of the invention also may use a flat sheet filter instead of a hollow fiber filter. Peristaltic pumps suitable for such as system are also commercially available.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof.

### DESCRIPTION OF THE FIGURES

FIG. 1 shows a schematic of an exemplary Tangential Flow Filtration system used in methods of the invention for concentrating and washing mammalian cells.

FIG. 2 illustrates the effects of shear rate on cell viability and recovery in the process of the invention.

FIG. 3 illustrates the effect of transmembrane pressure (TMP) on cell viability and recovery in the process of the invention. Panel A. Viability. Panel B. Recovery. Panel C. Trend curve of TMP vs Cell Recovery and Viability for multiple experiments.

FIG. 4 illustrates the effect of filter pore size on flux and cell viability and recovery in the process of the invention.

FIG. 5 illustrates concentration of cells according to the process of the invention.

FIG. 6 illustrates removal of undesirable components according to the process of the invention.

FIG. 7 illustrates cell quality (viability) after processing according to the invention.

FIG. 8 illustrates functionality (proliferation) of cells processed according to the process of the invention.

FIG. 9 illustrates flux and TMP during a large scale run (25 L of harvested cells) of the process of the invention.

FIG. 10 illustrates viability and cell recovery during a large scale run (25 L of harvested cells) of the process of the invention.

FIG. 11 illustrates use of a flat sheet filter in concentration of cells according to the process of the invention, including Viability and Total Cell Density (TCD) as a function of time during cell concentration.

FIG. 12 illustrates use of a hollow fiber filter in concentration of cells according to the process of the invention under conditions similar to those for the flat sheet filter in FIG. 11.

FIG. 13 illustrates use of concentration of CHO cells according to the process of the invention under conditions similar to those in FIG. 12, including Viability and Total Cell Density (TCD) as a function of time during cell concentration.

### DETAILED DESCRIPTION OF THE INVENTION

The present invention provides improved methods, and associated apparatus and systems for concentration and washing of live mammalian cells, particularly for preparation of human cell therapy products.

The present invention relates to a system and a method for aseptically processing live mammalian cells in an aqueous medium to produce a cell suspension having a cell density of at least about 5 million cells/mL and cell viability of at least about 70%, the method comprising a step of reducing the volume of the medium using a tangential flow filter (TFF) having a pore size of greater than 0.1 micron, wherein during the step the trans-membrane pressure (TMP) is maintained at less than about 3 psi and the shear rate is maintained at less than about 4000 sec⁻¹.

The cell viability using the present method is at least about 70% and can be 80%, 90% or more.

The shear rate of the method is maintained at less than about 3000 sec⁻¹ and the TMP is maintained at less than about 1 psi. The pore size of the TFF is about 0.65 micron and the TFF is a hollow fiber filter having a filtration surface area of at least about 0.5 ft².

The flux rate across the filter of the method is at least about 50 L/m² h, and can also at least about 300 L/m² h.

Where the recovery of the cells in the cell suspension is at least about 70% of the cells in the aqueous medium, the recovery is determined as a percentage of starting cell number versus the final cell number. In the present method, the cell suspension contains between about million to about 75 million viable cells/mL or between about 10 million viable cells to about 200 million viable cells/mL. Additionally, the viability of the cells in suspension is at least about 70%, and typically at least 80%, or 90% or more.

in another embodiment, the method comprises a diafiltration step wherein the TFF is used to wash the cells in the suspension with a volume of an aqueous wash medium equal to at least about 4 times the volume of the cell suspension. The residual level of an undesirable soluble component in the cell suspension is reduced by at least about 1000 fold compared to the level in the aqueous medium and can be reduced to less than about one part per million of the cell suspension.

In yet another embodiment of the invention, the comprising measuring a viable cell concentration using a sensor. The method provides a feedback mechanism to control TFF processing, identify when the certain steps in the process should end and another process step to begin (e.g., end concentration and begin diafiltration), and eliminate the need for verification sampling and thereby prevents risk of contamination during cell processing. The method thus comprises detecting a ‘real time’ signal from the sensor, wherein sampling during TFF is eliminated, processing the signal to determine processing stage, providing feedback to end TFF process steps when a target density of viable cells is reached. The signal measured by the sensor used in the method is transmitted through an amplifier sensor into a human machine interface, the signal can be converted into a viable cell density (VCD) data. The further comprising analyzing the VCD data to determine optimum final cell density and/or concentration factor. This VCD data can also be coupled with total cell density (TCD) sensors to calculate total cell viability (TCV), which is another critical quality parameter from which process decisions can be made.

In yet another embodiment of the invention is provide a method of manufacturing cells for use in a therapeutic composition, the method comprising the steps of expanding the cells using large scale cell cultures; harvesting the cells in a aqueous medium, reducing the volume of the cells in the aqueous medium and washing the cells using a TFF as disclosed herein, formulating the resulting cell suspension in a cryoprotective medium, and freezing and storing the formulated cells under conditions suitable for long-term maintenance of cell viability, wherein the frozen formulated cells exhibit the following parameters: cell viability on thawing of at least about 80%; viable cell density greater than about 5 million cells/mL; and residual levels of an undesirable soluble component in formulated cells is reduced to a level of less than about 1 ppm.

Another embodiment of the invention comprises a system including apparati comprising cell factories, bioreactors, tanks, to practice the method as disclosed herein.

To address the challenge of processing large batches of cells harvested from cultures in reasonable time (e.g., about 10 to about 100 L in less than 3 hrs), the inventors have developed a completely closed, fully disposable and scalable Tangential Flow Filtration (TFF) System using, for instance, a Hollow-Fiber Filter (HFF). This TFF system can process (separate, clarify, recover and collect cells from the fluid media) large volume batches in less than 3 hrs while maintaining high cell viability and functionality. In this system the HFF has aseptic quick connectors attached such that system assembly simply requires three quick connections and sterile welds of plastic tubing (depending on batch size).

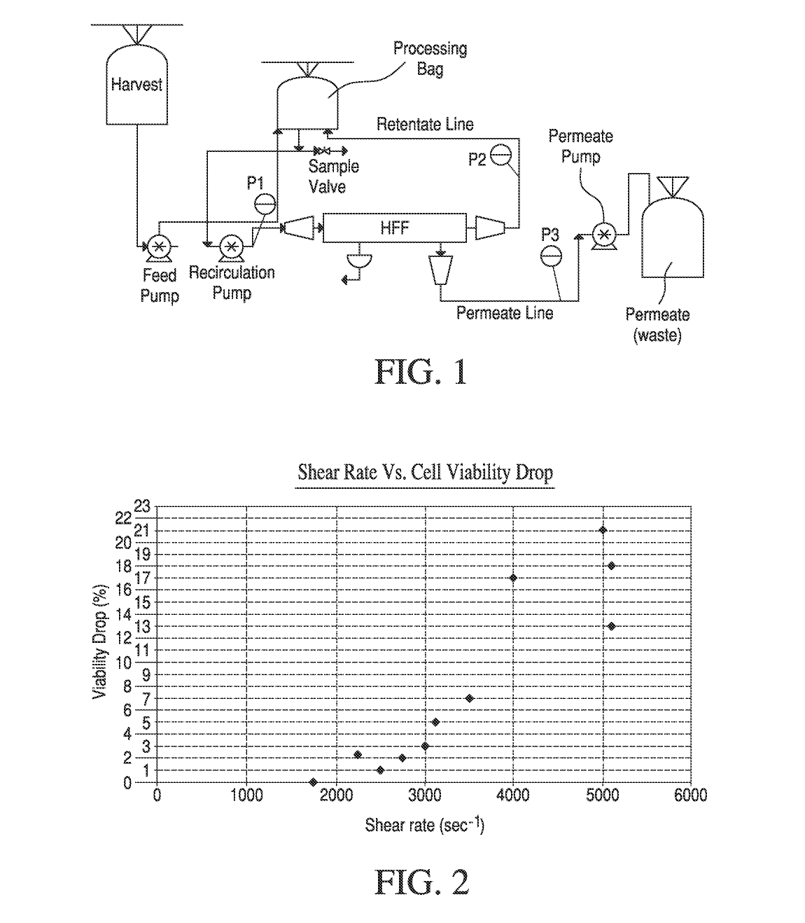

FIG. 1 below shows an exemplary TFF system of the present invention, which is completely closed and fully disposable. The tubing, disposable sensors (P1, P2, and P3 in FIG. 1) and processing reservoir (FIG. 1) were custom assembled and sterilized via gamma radiation. The disposable filter was sourced from GE (RTPJCFP-6-D4M, RTPCFP-E-4M and PN:RTPCFP-6-D-5) and assembled aseptically. Multiple TFF runs were completed with this or similar set-ups. In addition, feasibility studies have shown that TFF system of the invention also may use a flat sheet filter instead of a hollow fiber filter.

A typical TFF process of the invention consists of two stages: volume reduction and diafiltration. During the volume reduction step the bulk volume (cell culture media) is filtered out through the permeate side of the filter until a desired cell concentration is reached in the processing bag as shown in FIG. 1. In a diafiltration stage following the volume reduction stage, the concentrated cells are washed with a fluid, such as a buffer, to remove cell culture or harvest media components that are undesired or unacceptable for human administration. Further volume reduction may also be carried out after diafiltration, to reach a desired cell density for formulation of the therapeutic product.

In another embodiment of the invention, as shown in FIG. 1, the closed and fully disposable TFF system as disclosed herein further induces a hollow fiber filter and Viable Cell Density (VCD) flow through sensor and detection (FT Sensor). The VCD FT Sensor may be preferably joined on the joined on the alternate line (S1). The use of the disposable VCD sensor eliminates product sampling during the TFF, thus minimizing risks and improving product quality and safety. Signal from the VCD FT Sensor can be transmitted with a human machine interface, e.g., a Human Machine Interface by FOGALE-SEMICON. The VCD data can thus be used to determine optimal cell density and/or concentration factor during TFF operation.

Membrane filtration processes generally fall within the categories of reverse osmosis, ultrafiltration, and microfiltration, depending on the pore size of the membrane. See, for instance, the '294 patent, supra. Conventionally, ultrafiltration employs membranes rated for retaining solutes between approximately 1 and 1000 kDa in molecular weight, reverse osmosis employs membranes capable of retaining salts and other low molecular weight solutes, and microfiltration, or microporous filtration, employs membranes in the 0.1 to 10 micrometer (micron) pore size range, typically used to retain colloids and microorganisms. Id. In TFF, the feed stream is recirculated at high velocities tangential to the plane of the membrane to increase the mass-transfer coefficient for back diffusion. The fluid flowing in a direction parallel to the filter membrane acts to clean the filter surface continuously and prevents clogging by non-filterable solutes. Id. In TFF, a pressure differential gradient, called transmembrane pressure (TMP), is applied along the length of the membrane to cause fluid and filterable solutes to flow through the filter. Flux is independent of TMP above a certain minimum value that can be determined empirically. Id. To achieve maximum flux, ultrafiltration systems are typically run with an outlet pressure equal to or greater than this minimum value. Hence, flux is constant along the length of the membrane, while the TMP varies.

Surprisingly, the relatively low sheer and TMP parameters of the TFF process of the present invention do not result in “clogging” of the filter and, instead, produce highly desirable transmembrane flux rates, such as at least about 100 L/m² h, preferably at least about 200 L/m² h, and more preferably at least about 300 L/m² h. Such flux rates greatly reduce the processing time for cell batch volumes of about 10 L to about 100 L, or even larger batches up to about 1000 L, compared to conventional centrifugation methods, thereby maintaining high cell quality including viability. Further, the above TFF process parameters of the invention surprisingly provide excellent recovery of cells in the resulting cell suspension, for instance, at least about 80%, preferably at least about 90%, more preferably at least about 95% or 99%, of the starting cells in the aqueous medium. Depending on the type of cell and the intended use, the resulting cell suspension may contain at least about 3 million, 6 million or even at least about 10 million viable cells/mL.

It will be readily apparent to one of ordinary skill in the relevant arts that other suitable modifications and adaptations to the methods and applications described herein are obvious and may be made without departing from the scope of the invention or any embodiment thereof. The present invention will be more clearly understood by reference to the following examples, which are included herewith for purposes of illustration only and are not intended to be limiting of the invention.

### EXAMPLES

During the development of this TFF procedure in the following Examples, primary human cells (typically adult mesenchymal stem cells or human dermal fibroblasts) were cultured in “Cell Factories” (Corning or Nunc®) and harvested into multiple bags (Lonza). Prior to the start of the experiment, bags of harvested cells were attached to the processing bag via sterile welding of plastic tubing or other aseptic connections (FIG. 1) and the cell suspension was transferred into the processing bag using a feed pump (Masterflex L/S) as shown in FIG. 1. The processing bag was filled with cell suspension to one half to two thirds of the bag capacity. The recirculation pump (Watson-Marlow-323E or Spectrum Krosflow) (as shown in FIG. 1) was started slowly and ramped up to the intended speed. Then permeate pump (Masterflex L/S) (as shown in FIG. 1) was started slowly and ramped to the intended speed. The feed pump and permeate pump were operated at the same speed using the same size tubing ( 3/16″ or ¼″ ID) to maintain a constant volume of fluid in the processing bag during the volume reduction step of the process. The recirculation pump typically used a ¼″ or ⅜″ ID tubing and was operated at constant speed to achieve desirable inlet flow rates. Cell suspension samples were collected periodically to measure cell density and viability using Nucleocounter (New Brunswick Scientific) or other cell counting techniques.

Typically, when the bulk volume was reduced to within about 100-800 mL (depending on desired cell concentration), diafiltration was started by attaching buffer bags to the processing bag. The cells were washed with 8-10 volume equivalents of a diafiltration buffer to achieve a reduction in residual culture and harvesting components. The volume in the processing bag may then be further reduced by filtration as described above, to achieve a desired cell concentration for formulation. When the desired cell concentration was achieved all pumps were stopped, and cell suspension from the filter and tubing were drained into the processing bag by gravity. A final sample from the processing bag was obtained for cell count, and the cell suspension was formulated and filled into vials for cryopreservation.

The critical quality parameters of a cell therapy process for 10-100 L of harvested cell suspension include: maintaining cell viability of at least 90% and high cell functionality, while concentrating cells to greater than 10 million cells/mL; yielding overall cell recovery (defined as (cells in−cells out)/cells in) of at least about 85%; reducing culture medium residuals such as bovine serum albumin (BSA) to less than 1 ug/mL (see, e.g., 21 CFR 610.15); and maintaining cell processing times that limit cell death, for instance, less than about 3 hours.

### Example 1

### Effects of Shear Rate

High shear rates are common in TFF processes as they help to minimize filter fouling (keeping cells from adsorbing onto the filter material), thus maximizing flux and minimizing processing times. The goal of this set of experiments was to optimize the shear rates while maintaining the critical quality parameters of the final cell suspension. Small scale (3-5 L) experimnents were performed according to the typical TFF procedure, above, to investigate the effect of shear rate on TFF process performance and product quality. Shear rate was calculated as

$$r = \frac{4 q}{π {R}^{3}} ,$$

where r is the shear rate (units=sec⁻¹), q is the filter inlet flow rate through the fiber lumen (units=m³/sec) and R is the fiber radius (in meters). With any given apparatus including specified filters and tubing, shear rate was controlled by the filter inlet flow rate (i.e., flow rate of Feed pump in FIG. 1), Approximately 2-3 billion human dermal fibroblasts were harvested from 2-3 cell factories (40-Layer Nunc) and processed with a 0.5 ft² size filter (GE PN:RTPCFP-6-D-4M). The cell concentrations at the beginning of the experiments ranged from 2.5×10⁵-5×10⁵ cells/mL and volumes were concentrated according to the typical TFF process for 62±9 minutes. Processing was performed in hollow fiber filters and in some cases (excessive shear rate) the diafiltration was not performed. The data in Table 1 demonstrate that high shear rates negatively affected cell viability.

TABLE 1

| | Final Cell | Final Cell | Average | |
|---|---|---|---|---|
| Shear Rate | Viability | Recovery | Flux | Processing |
| (sec⁻¹) | (%) | (%) | (LMH) | Time (min.) |
| 5100 | 70 | 90 | 130 | 62 |
| 1750 | 95 | 100 | 100 | 53 |
| 2500 | 96 | 95 | 115 | 70 |

Trending analysis of shear rates over several experiments demonstrated the effect of shear on the viability of the final concentrated cell product (FIG. 2), in which “viability drop” is defined as the difference in pre-TFF and post-TFF cell viabilities as measured by Nucleocounter assay.

Conclusion: This set of studies demonstrates that final cell viability is dependent on the fluid shear rate of the process, which should preferably be maintained below about 3000 sec⁻¹.

### Example 2

### Effect of TMP on the Quality Parameters of Cell Suspensions

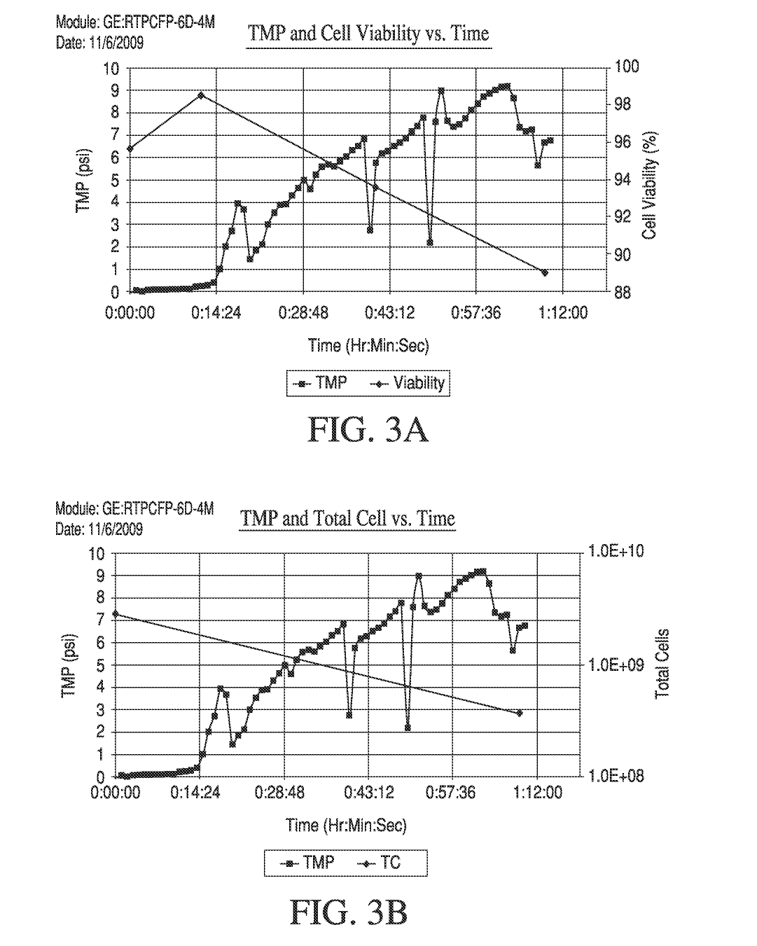

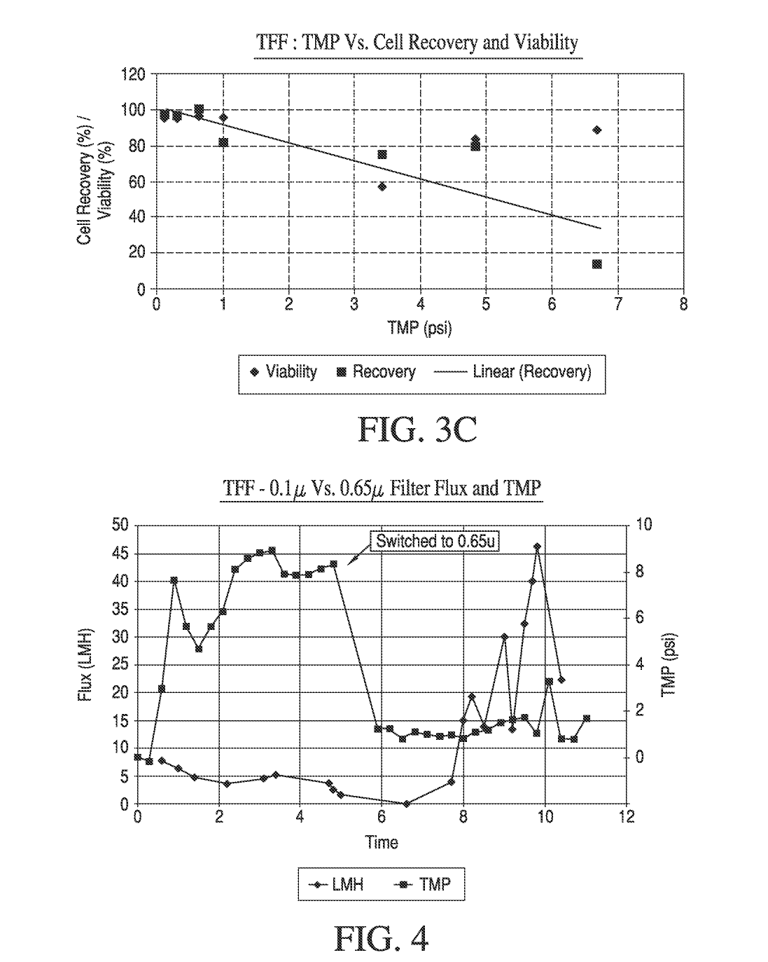

Trans-membrane pressure (TMP) is a critical processing variable for TFF. During processes without permeate control (such as in the '669 patent, above), TMP is the main driving force for permeate flow and controls flux rates. Thus, high TMPs drive high flux rates and low processing times. We therefore set out to determine the effect of TMP on the quality parameters of processed cells using the specified apparatus. For this experiment approximately 2.9 billion human Dermal Fibroblast (hDF) cells were harvested from cell factories (40-Layer Nunc) and processed with a 0.5 ft² size hollow fiber filter (GE PN:RTPCFP-6-D-4M) at a relatively high shear rate of 3500-3900 sec⁻¹. This high shear rate was chosen to minimize filter fouling while maximizing cell viability. TMP was calculated as

$$TMP = \frac{{P}_{1} + {P}_{2}}{2} - {P}_{3}$$

where P₁ is the filter inlet pressure, P₂ is the filter outlet pressure and P₃ is the permeate pressure. For a given apparatus, TMP was controlled by varying P₁, P₂, and/or P₃ via controlling pump flow rates of the Recirculation and Permeate pumps shown in FIG. 1. The cell concentration at the beginning of the experiment was 4.2×10⁵ cells/mL, and cells were concentrated essentially according to the typical process above. FIGS. 3a and 3b show the affect of moderate TMP levels (5-10 psi) on TFF process performance and product quality.

During processing, as TMP increased from filter clogging to over 5 psi, both viability and cell recovery were impacted. Cell viability dropped below 90% during the volume reduction step (other experiments where diafiltration was performed have demonstrated that viability can drop to less than 80% if processing continues), which may have been due to the shear rate during processing. However, importantly almost 90% of the cells were lost during processing due to the high transmembrane pressures which drove filter fouling, reducing the flux rate to almost zero.

Conclusion: During this experiment the cell viability decreased below 90% (during the volume reduction step only, diafiltration could not be performed due to poor process performance) and a significant portion (88%) of the cell product was lost. Furthermore, the ability to obtain cell suspensions for formulation of >10 M/mL was unattainable (final concentration was only 0.5 M cells/mL) due to the filter fouling and poor cell recovery. Thus this experiment demonstrates that TMPs greater than 5 psi negatively impact cell viability, final cell concentration, and cell recovery. This underlies the importance of balancing processing time (via flux) with other processing parameters in order to maintain the quality attributes of a therapeutic cell product.

Further studies showed that acceptable (at least 90%) recoveries are obtained with TMP equal to or less than about 1 psi, whereas TMPs above about 1 psi up to 5 psi had a moderate impact on cell recovery, and TMPs over 5 psi had a significant negative effect on cell recovery. FIG. 3c is a trending chart of TMP (average during the run), cell recovery and cell viability from multiple TFF experiments.

### Example 3

### Effect of Pore Size on Flux

Small scale (3-5 L) experiments were performed to investigate the effect of filter pore size on TFF process performance and product quality. Approximately 3 billion human dermal fibroblasts were harvested from cell factories (40-Layer Nunc) and processed with 0.5 ft² size hollow fiber filters of 0.65 micron pores (GE PN:RTPCFP-6-D-4M) or 0.1 micron pores (RTPCFP-1-E-4M). The cell concentrations at the beginning of the experiments ranged from 4×10⁵-×10⁵ cells/mL and were concentrated essentially according to the typical process, above. Table 2 below provides range of filter pore size tested and corresponding product quality and process performance parameters.

TABLE 2

| Filter | | | | | |
|---|---|---|---|---|---|
| pore | Final Cell | Final Cell | Average | Average | |
| size | Viability | Recovery | TMP | Flux | Processing |
| (micron) | (%) | (%) | (psi) | (LMH) | Time (min.) |
| 0.1ᵤ | 95 | N/A* | 8 | 47 | N/A* |
| 0.65ᵤ | 96 | 95 | 1 | 280 | 30 |

The experiment with the 0.1 micron pore filter could not be completed due to poor permeate flow rate (flux). Thus cell recovery and processing time data are not applicable (“N/A*”).

FIG. 4 shows the flux rate and TMP of the experiments performed to investigate the effect of filter pore size on TFF process performance.

Conclusion: this set of studies demonstrates that reduction of pore size from 0.65 micron to 0.1 micron leads to filter fouling, decreasing flux, increasing TMP which eventually has a negative impact on the critical quality parameters of the final cell product, often leading to product failure (viability <80%).

### Example 4

### Effects of High Flux Rate

Small scale (3-5 L) experiments were performed to investigate the effect of high flux rates on TFF process performance and product quality. Approximately 3 billion human dermal fibroblasts were harvested from cell factories (40 Layer Nunc) and processed with a 0.5 ft² size hollow fiber filter (GE PN:RTPCFP-6-D-4M). The cell concentrations at the beginning of the experiments ranged from 2.5×10⁵-5×10⁵ cells/mL and were concentrated essentially according to the typical process, above. Flux was controlled by increasing the permeate flow rate while maintaining the recirculation rate. Table 3 below provides the range of flux rates achieved and corresponding product quality parameters. Flux ranged from 150-350 LMH and shear rates ranged from 2000 to 3000 sec⁻¹.

TABLE 3

| Average Flux | Final Cell | Final Cell |
|---|---|---|
| (LMH) | Viability (%) | Recovery (%) |
| 150-200 | 91 | 99 |
| 150-350 | 96 | 91 |

Conclusion: this set of studies demonstrates that the flux (in LMH) can be achieved in the range of 150-350 LMH using permeate flow rate control while minimizing shear rates and TMP, and maintaining cell viability of at least 90%, and cell recovery of at least 90%. This high rate of flux allows for minimization of processing time for large volumes, which has an overall positive impact on product quality.

### Example 5

### Large Scale Bioprocessing of Human Mesenchymal Stem Cells (hMSCs)

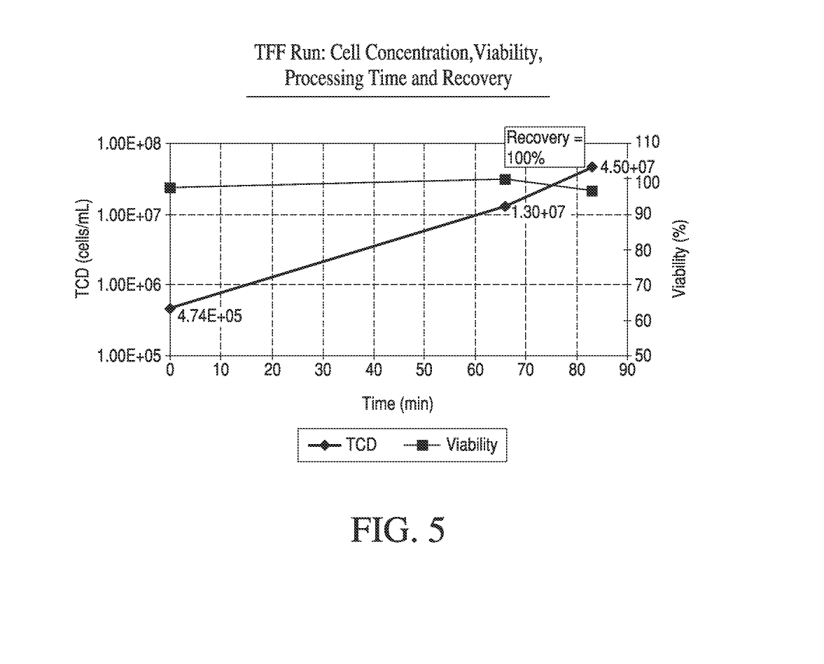

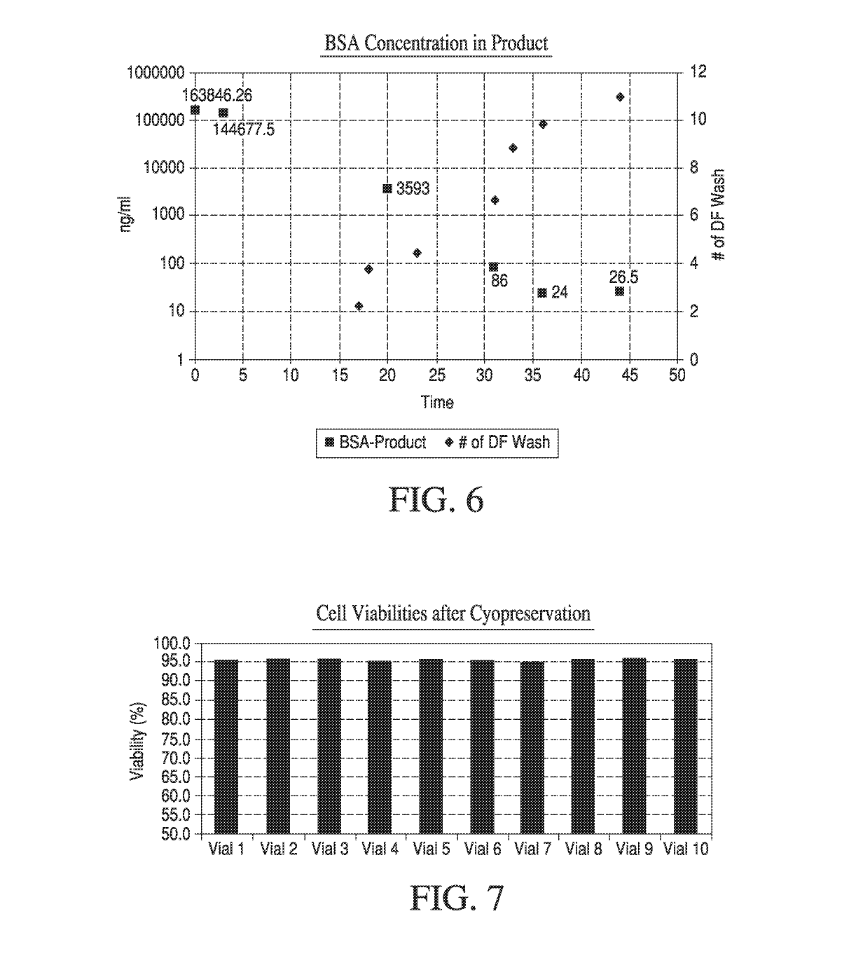

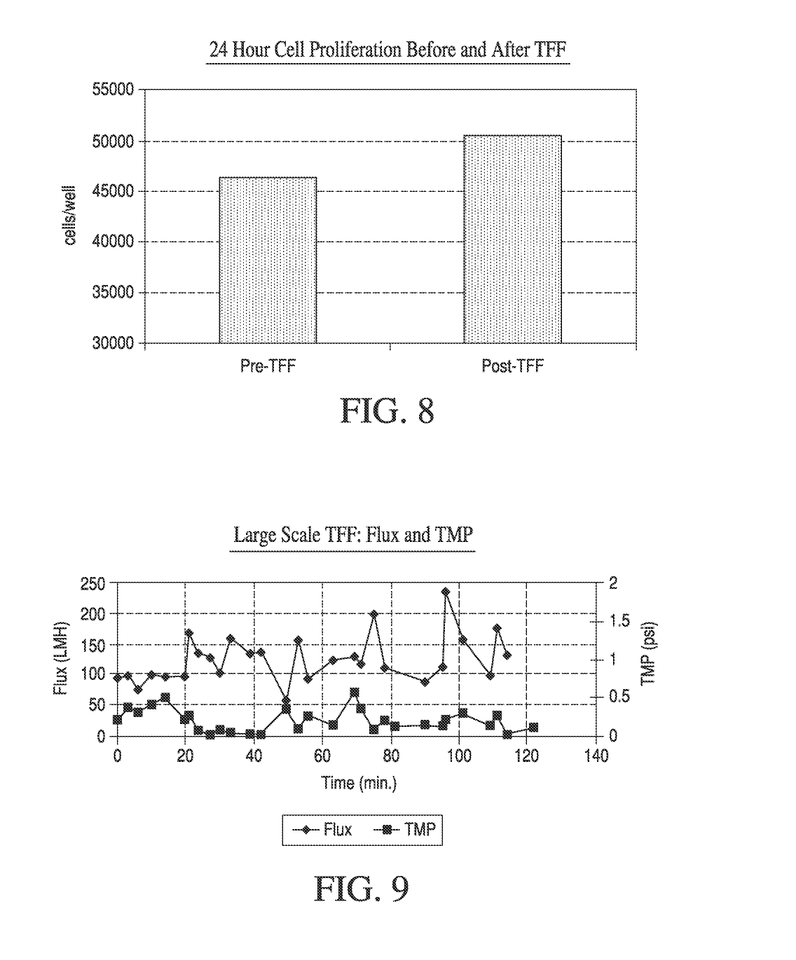

Using the above typical procedure and the TFF system in FIG. 1, human mesenchymal stem cells (MSCs from Lonza Bioscience) were expanded using multiple Nunc or Corning 40 layer culture vessels, and concentrated from a harvested concentration of 2-4×10⁵ cells/mL up to 4×10⁷ cells/mL, while maintaining cell viability of about 95% and recovering essentially all of the cells from the process. See FIG. 5. After concentration and difiltration using 8-10 diafiltration volumes of physiological saline with human serum albumin (HSA), the concentrated cells had final BSA residual levels reduced to less than 100 ng/mL (FIG. 6). The cells were then further processed using scalable single use systems. After volume reduction and washing, the hMSCs were counted using a Nucleocounter to establish concentration and viability, to establish TFF processing yields as well as to guide formulation. The cells were formulated to achieve a final solution of 7.5% dimethylsulfoxide (DMSO) in ProFreeze™ (Lonza) at ˜8-12 M cells/mL. The formulated hMSCs were the dispensed into 20 mL vials using a Flexicon® pump and a single use tubing set connected to the formulation bag. The vials were frozen in bulk (˜200 vials to simulate a large scale freeze) in a controlled rate freezer, and were stored for 7-14 days in vapor phase liquid nitrogen. Upon thawing, cell viability (FIG. 7) and viable cell recovery was maintained well over 90% in the final container. To asses whether the TFF process had any impact on the general health and viability of processed cells, processed and unprocessed cells were seeded at 30,000 cells per well in a 96 well plate, and 24 hour viability and cell functionality were monitored using the Vialight® (Lonza) cell metabolism and viability assay. Importantly, the TFF process had little to no impact on the cells harvested from the large scale (40 layer) culture platform (FIG. 8).

This set of studies demonstrates that clinically relevant cells (namely, human MSCs) which have been expanded and harvested in large scale culture vessels may be successfully concentrated and washed using the TFF processes of the present invention, removing residual proteins while maintaining the critical quality parameters such as viability and yield of the final cell product, even after cryopreservation. These cells maintain biological functionality in culture when compared to unprocessed cells, and may be cryopreserved at high concentrations with excellent recovery after processing. In summary, these data demonstrate that TFF is a reliable method of volume reduction and washing for large scale cell therapy manufacturing.

### Example 6

### Large Scale (25 L) Processing Run

This experiment was performed to assess the TFF process performance and product quality at large scale (25 L). For this experiment approximately 18 billion CHO cells were harvested from multiple shake flasks (Corning®) and processed with a 1.7 ft² size hollow fiber filter (GE PN:RTPCFP-6-D-5). The cell concentrations at the beginning of the experiments ranged from 2.5×10⁵-5×10⁵ cells/mL and were concentrated according to the typical process, above, using the TMP and flux parameters shown in Table 4 which also shows the product quality and process performance parameters.

TABLE 4

| Process | Final Cell | Final Cell | | Flux | |
|---|---|---|---|---|---|
| Volume | Viability | Recovery | TMP | Range | Processing |
| (L) | (%) | (%) | (psi) | (LMH) | Time (min.) |
| 25 | 96 | 97 | 0-0.6 | 100-235 | 128 |

FIG. 9 shows the flux and TMP of the experiment performed to assess the TFF process performance and product quality at large scale (25 L).

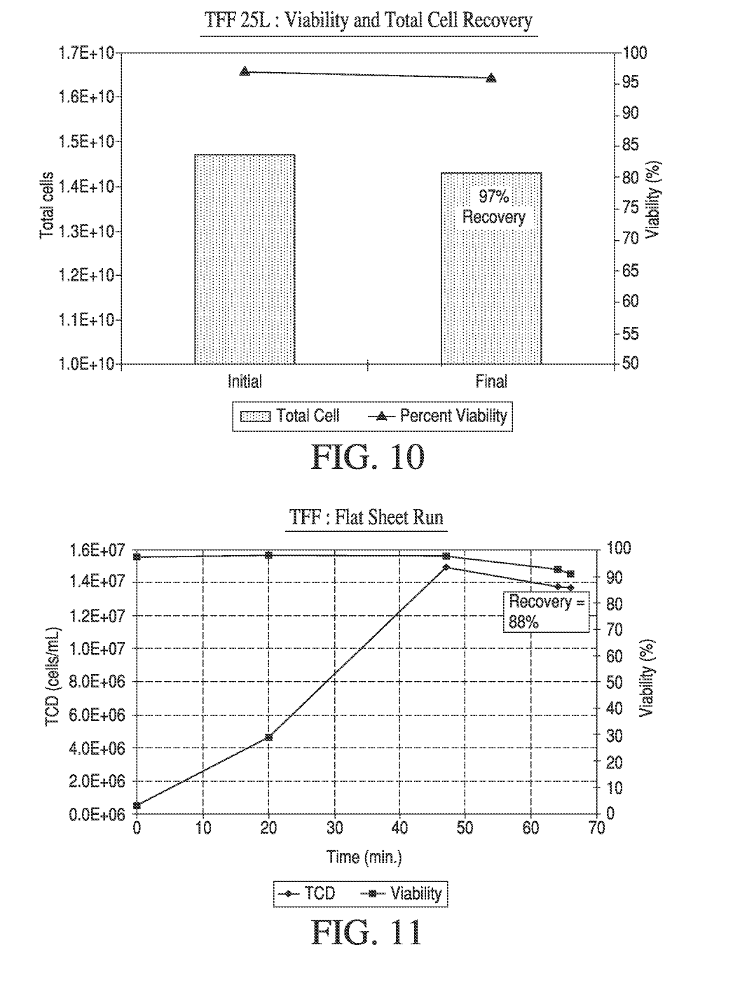

FIG. 10 shows the cell viability and recovery of the experiment performed to assess the TFF process performance and product quality at large scale (25 L).

Conclusion: this study demonstrates the scalability of the process to commercial scale while maintaining the critical quality parameters of the final cell product.

### Example 7

### Comparison of Hollow Fiber and Flat Surface Filter Types

Small scale experiments were performed to investigate the feasibility of using flat sheet filters to process cells. For these experiments approximately 1-3 billion mesenchymal stem cells or human dermal fibroblasts were harvested from 8-12 10-Layer cell factories or 1-3 40-Layer cell factories and processed with either hollow fiber filters (0.5 ft² or 1 ft² size filter (GE PN:RTPCFP-6-D-4M)) or flat sheet filters ranging from 0.1 to 0.65 pm from Sartorius and Pall. The cell concentrations at the beginning of the experiments ranged from 4.5×10⁵-5×10⁵ cells/mL. The cells were concentrated essentially according to the process in Example 1.

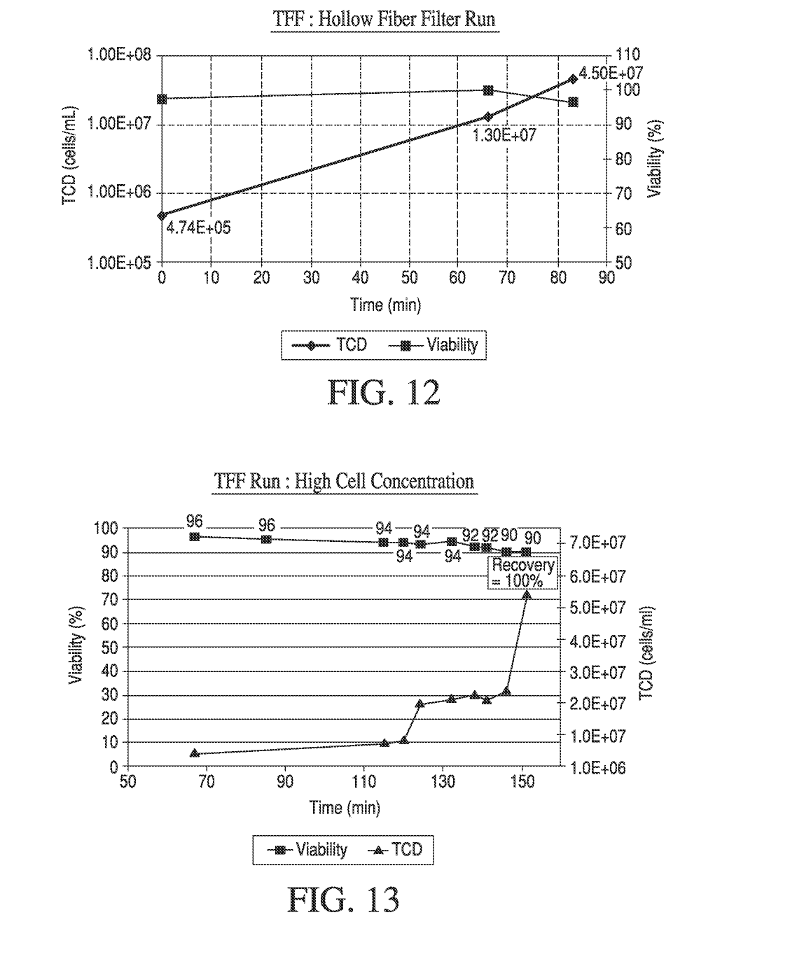

FIG. 11 shows an example of a flat sheet run and FIG. 12 shows an example of a hollow fiber filter run.

Conclusion: this set of studies demonstrates that the type of TFF platform, hollow fiber or flat sheet, does not substantially impact the critical quality parameters of the system, and that cell viability and cell recovery can be achieved according to the invention, regardless of TFF platform.

### Example 8

### Final Cell Concentration

Small scale (3-5 L) experiments were performed to investigate the feasibility of achieving high cell concentrations using TFF process. Approximately 5 billion human dermal fibroblasts were harvested from 4-5 cell factories (40-Layer Nunc) and processed with a 0.5 ft² size hollow fiber filter (GE PN:RTPCFP-6-D-4M). In a second experiment, approximately 10 billion Chinese Hamster Ovary (CHO) cells were harvested from multiple shake flasks and processed with an identical 0.5 ft² size hollow fiber filter. The cell densities at the beginning of the experiments ranged from about 4×10⁵-5×10⁵ cells/mL, and cells were concentrated essentially according to the typical process, above. FIG. 13 shows Total Cell Density (TCD) achieved as a function of time during the course of the CHO experiment, and Table 5 provides final TCDs achieved and corresponding product quality parameters for both experiments.

TABLE 5

| Final Total Cell | Final Cell | Final Cell |
|---|---|---|
| Density | Viability | Recovery |
| (cells/mL) | (%) | (%) |
| 45 × 10⁶ | 97 | 100 |
| 54 × 10⁶ | 90 | 100 |

Conclusion: this set of studies demonstrates that cells may be concentrated at least to densities greater than 50 million/mL, while maintaining cell viability and recovery. In other similar experiments, TCDs in excess of 100 million cells/mL have been achieved by the present invention method.

### Example 9

### Filter Loading Capacity

For these studies hMSC cells were harvested from 1-2 Cell Factories (40-Layer Nunc) and CHO cells from multiple shake flasks. The hMSC cells were processed with a 0.5 ft² hollow fiber filter (GE PN:RTPCFP-6-D-4M) and the CHO cells were processed with a 1.7 ft² hollow fiber filter (GE PN:RTPCFP-6-D-5). The cell concentrations at the beginning of the experiments ranged from 2.5×10⁵-5.6×10⁵ cells/mL and were concentrated according to the process in Example 1.

Table 6 below provides the range of cells per area of filter processed and corresponding product quality parameters.

TABLE 6

| Total Cells per | Final Cell | Final Cell | |
|---|---|---|---|
| area of filter | Viability | Recovery | TMP |
| (cells/ft²) | (%) | (%) | (psi) |
| 1.4 × 10⁹ | 96 | 98 | <1 |
| 8.6 × 10⁹ | 96 | 97 | <1 |

Conclusion: this set of studies demonstrates that filters may be loaded as low as 1.4 billion cells/ft² and as high as 8.6 billion cells/ft², while maintaining acceptable cell viability and recovery. In addition, theoretically the filter load may be at least as low as 0.75 billion cells/ft² while maintaining acceptable process performance parameters.

It will be understood that the embodiments of the present invention which have been described are illustrative of some of the applications of the principles of the present invention. Numerous modifications may be made by those skilled in the art without departing from the true spirit and scope of the invention.

### Example 10

### TFF System with FT Sensor

FIG. 1 shows a completely closed and fully disposable TFF system with hollow fiber filter and Viable Cell Density (VCD) flow through sensor/detector (FT Sensor) that was designed at Lonza. The tubing, disposable sensors (P1, P2, P3 and S1 in FIG. 1) including the VCD sensor and processing reservoir (FIG. 1) were custom assembled and sterilized via gamma radiation. The use of the disposable VCD sensor eliminates product sampling during the TFF thus minimizes risks and improves product quality and safety. The disposable filter was sourced from GE (RTPCFP-6-D-4M, RTPCFP-1-E-4M and PN:RTPCFP-6-D-5) and assembled aseptically. Multiple TFF runs were completed with this or a very similar set-up. In addition, TFF system may use a flat sheet filter instead of a hollow fiber filter. Feasibility studies were performed with flat sheet filters. A typical TFF process consists of two steps-volume reduction and diafiltration. During the volume reduction step the bulk volume (cell culture media) is filtered out through the permeate side of the filter until a desired cell concentration is reached in the processing bag as shown in FIG. 1. Diafiltration step follows the volume reduction step. During the diafiltration the concentrated cells are washed with a buffer to remove impurities.

For a typical TFF experiment, primary human cells (mostly mesenchymal stem cells or human dermal fibroblasts) are cultured in cell factories (Corning or Nunc®) and harvested into multiple bags (Lonza). Prior to the start of the TFF experiment, bags with harvested cells are attached to the processing bag via sterile welding or other aseptic connections (FIG. 1) and the cell suspension is transferred into the bag using a feed pump (Masterflex L/S) as shown in FIG. 1. Signal from the VCD FT Sensor is transmitted through an amplifier cable into a Human Machine Interface (HMI) from Fogale. The signal is converted to VCD measure and displayed on the HMI. This VCD data is used to determine the optimum final cell density and/or concentration factor during the TFF operation. The processing bag is filled with cell suspension to one half to ⅔rd of the bag capacity. The recirculation pump (Watson-Marlow-323E or Spectrum KrosFlow) (as shown in FIG. 1) is started slowly and ramped up to the intended speed. Then permeate pump (Masterflex L/S) (as shown in FIG. 1) is then started slowly and ramped to the intended speed. The feed pump and permeate pump are operated at the same speed using the same size tubing ( 3/16″, ¼″ or ⅜″ID) to maintain a constant volume of fluid in the processing bag during the volume reduction step of the process. The recirculation pump typically used a ¼″ or ⅜″ ID tubing and is operated at constant speed to achieve desirable inlet flow rates. Cell suspension samples may be collected periodically to measure cell density and viability using Nucleocounter (NC) (New Brunswick Scientific) or other cell counting techniques. Typically once the bulk volume is reduced to within 100-800 mL based on the VCD data from the VCD FT Sensor the diafiltration step is started by attaching buffer bags to the processing bag. The cells are washed with 8-10 volume equivalents of a diafiltration buffer to achieve a reduction in residual culture and harvest reagents. The volume in the processing bag may be reduced further to achieve a desired cell concentration based on the VCD data from the VCD FT Sensor for formulation. Once desired cell concentration is achieved all pumps are stopped. Cell suspension from the filter and tubing are drained into the processing bag by gravity. A final sample from the processing bag is obtained for cell count and cell the suspension is formulated and filled into vials for cryopreservation.

The critical quality parameters of a cell therapy process must maintain cell viability >90% while concentrating cells to greater than 5M cells/nm, and cell functionality must be maintained.

This set of structures show (FIG. 13) that mesenchymal stem cells can be concentrated from 2-4×10⁵ to up to 0.3×10⁷ cells/mL and the VCD sensor signal (permittivity) correlates with the increasing VCD that is measured using Nucleocounter method. Importantly, this process may be performed in a completely disposable single use set up.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

What is claimed is:

1. A method for aseptically processing live mammalian cells in an aqueous medium to produce a cell suspension having a cell density of at least about 5 million cells/mL and cell viability of at least about 70%, the method comprising a step of reducing the volume of the medium using a tangential flow filter (TFF) having a pore size of greater than 0.1 micron, wherein during the step the trans-membrane pressure (TMP) is maintained at less than about 3 psi and the shear rate is maintained at less than about 4000 sec⁻¹.

2. The method of claim according to claim 1, wherein the cell viability is at least about 80%.

3. The method according to claim 1, wherein the cell viability is at least about 90%.

4. The method according to claim 1, wherein the shear rate is maintained at less than about 3000 sec⁻¹.

5. The method according to claim 1, wherein the TMP is maintained at less than about 1 psi.

6. The method according to claim 1, wherein the pore size of the TFF is about 0.65 micron.

7. The method according to claim 1, wherein the TFF is a hollow fiber filter having a filtration surface area of at least about 0.5 ft².

8. The method according to claim 7, wherein the flux rate across the filter is at least about 50 L/m² h.

9. The method according to claim 7, wherein the flux rate across the filter is at least about 300 L/m² h.

10. The method of claim 1, wherein the recovery of the cells in the cell suspension is at least about 80% of the cells in the aqueous medium wherein the recovery is determined as a percentage of starting cell number versus the final cell number.

11. The method of claim 1, wherein the cell suspension contains between about 10 million to about 75 million viable cells/mL.

12. The method of claim 1 wherein the cell suspension contains between about 10 million viable cells to about 200 million.

13. The method of claim 1, wherein the viability of cells in the suspension is between about 90% and about 100%.

14. The method according to claim 1 further comprising a diafiltration step wherein the TFF is used to wash the cells in the suspension with a volume of an aqueous wash medium equal to at least about 4 times the volume of the cell suspension.

15. The method according to claim 14, wherein the residual level of an undesirable soluble component in the cell suspension is reduced by at least about 1000 fold compared to the level in the aqueous medium.

16. The method according to claim 14, wherein the residual level is reduced to less than about one part per million of the cell suspension.

17. The method according to claim 1, further comprising measuring a viable cell concentration using a sensor.

18. The method according to claim 1, further comprising measuring a total cell viability using a sensor.

19. The method of claim 17, further comprising detecting a signal from the sensor, wherein sampling during TFF is eliminated.

20. The method according to claim 18, further comprising processing the signal to determine processing stage.

21. The method according to claim 18, wherein the processing further comprises providing feedback to make processing assessing.

22. The method according to claim 13, wherein the signal is transmitted through an amplifier sensor into a human machine interface.

23. The method according to claim 14, further comprising converting the signal into a variable cell density (VCD) data.

24. The method according to claim 15, further comprising analyzing the VCD data to determine optimum final cell density and/or concentration factor.

25. A method of manufacturing cells for use in a therapeutic composition, the method comprising the steps of
expanding the cells using large scale cell cultures;
harvesting the cells in a aqueous medium;
reducing the volume of the cells in the aqueous medium and washing the cells using a TFF in the method of claim 11; and
formulating the resulting cell suspension in a cryoprotective medium, and freezing and storing the formulated cells under conditions suitable for long-term maintenance of cell viability,
wherein the frozen formulated cells exhibit the following parameters:
cell viability on thawing of at least about 80%;
viable cell density greater than about 5 million cells/mL; and
residual levels of an undesirable soluble component in formulated cells is
reduced to a level of less than about 1 ppm.

26. A product made by the method of claim 25.
